(19) United States
(12) Patent Application Publication
(10) Pub. No.: US 2026/0098956 A1
(43) Pub. Date: Apr. 9, 2026

(54) HUMAN BODY TRACKING DEVICE AND HUMAN BODY TRACKING METHOD

(71) Applicant: Murata Manufacturing Co., Ltd., Nagaokakyo-shi (JP)

(72) Inventors: Kosei ASANO, Nagaokakyo-shi (JP); Takahiro KINOSHITA, Nagaokakyo-shi (JP)

(73) Assignee: Murata Manufacturing Co., Ltd., Nagaokakyo-shi (JP)

(21) Appl. No.: 19/405,434

(22) Filed: Dec. 2, 2025

Related U.S. Application Data
(63) Continuation of application No. PCT/JP2024/026863, filed on Jul. 26, 2024.

(30) Foreign Application Priority Data
Oct. 5, 2023 (JP) 2023-173821

(51) Int. Cl. G01S 13/72; A61B 5/00; A61B 5/024; A61B 5/05; A61B 5/11; G01S 7/41; G01S 13/56

(52) U.S. Cl. CPC G01S 13/723; A61B 5/02444; A61B 5/05; A61B 5/1113; A61B 5/725; G01S 7/415; G01S 13/56

(57) ABSTRACT

A human body tracking device and a human body tracking method capable of improving the accuracy in tracking of a human body are implemented. The device includes a transmitter/receiver that transmits a radio wave and receives a reflected wave of the transmitted radio wave, and a processor that estimates a location of a human body on the basis of an intermediate frequency (IF) signal output from the transmitter/receiver. The processor is configured to execute a first detection process that detects coordinates of a moving object as first coordinates, a second detection process that detects coordinates of at least the human body in a stationary state as second coordinates, and a tracking process that tracks the human body on the basis of the first coordinates and the second coordinates.

### CROSS-REFERENCE TO RELATED APPLICATIONS

The present application is a continuation of PCT International Application No. PCT/JP2024/026863, filed on Jul. 26, 2024, which claims priority to Japanese patent application JP 2023-173821, filed Oct. 5, 2023, the entire contents of each of which being incorporated herein by reference.

### TECHNICAL FIELD

The present disclosure relates to a human body tracking device and a human body tracking method.

### BACKGROUND ART

Systems that detect a human body by using RAdio Detection And Ranging (RADAR) and acquire biological information (hereinafter, also referred to as a “vital sign”) based on a body surface displacement of the human body have been being introduced.

In a tracking system using radar, a reflected wave of a radio wave radiated from an antenna is analyzed so that a target can be identified. In Patent Document 1, a technique for extracting a vibration source including body movement of a person such as heart rate and performing beamforming of a transmission wave so that the vibration source can be tracked is disclosed. In Patent Document 2, a technique for identifying the type of a target on the basis of environmental information defined for each location is disclosed.

Furthermore, as a method for extracting a vital sign, a technique using the degree of correlation between the amplitude and phase of a signal obtained from a reflected wave of a Frequency Modulated Continuous Wave (FMCW) radar is disclosed (for example, Non Patent Document 1).

### CITATION LIST

### Patent Documents

Patent Document 1: Japanese Unexamined Patent Application Publication No. 2022-182179
Patent Document 2: International Publication No. 2018/211948

### Non Patent Document

Non Patent Document 1: H. Choi, H. Song, and H. Shin, “Target Range Selection of FMCW Radar for Accurate Vital Information Extraction”, IEEE Access, vol. 9, pp. 1261-1270, 2020.

### SUMMARY

### Technical Problems

In the technique described in Patent Document 1, periodic vibrations caused by disturbance may be detected as vital signs and reflection from a stationary object (hereinafter, also referred to as “clutter”) may be falsely determined to be reflection from a human body.

Furthermore, in the technique described in Patent Document 2, environmental information needs to be defined for each location. In other words, under the circumstances where environmental information is not defined, identification of a target cannot be achieved. Furthermore, if the state of a target (variations in displacement) has changed, specifically, for example, if a pedestrian as a target has stopped, the target may be lost.

As described above, in conventional FMCW (Frequency Modulated Continuous Wave) radar signal processing, a target is typically identified based on a Doppler shift caused by relative velocity. Consequently, when a human target transitions from a moving state to a stationary state (e.g., sitting down or standing still), the Doppler shift approaches zero. In this “zero-Doppler” state, the radar system struggles to distinguish the human body from other static objects (clutter), e.g., walls or furniture. Conventional methods to mitigate this require computationally expensive background subtraction or pre-mapped environmental data, which lack flexibility in dynamic environments.

The present disclosure has been designed in view of the problems mentioned above, and the present disclosure is directed to implementing a human body tracking device and a human body tracking method capable of improving the accuracy in tracking of a human body.

### Solutions to Problems

A human body tracking device according to an aspect of the present disclosure includes a transmitter/receiver that receives a reflected wave of a transmitted radio wave, and a processor that estimates a location of a human body on the basis of an intermediate frequency (IF) signal output from the transmitter/receiver. The processor includes a first detection unit that detects coordinates of a moving object as first coordinates, a second detection unit that detects coordinates of at least the human body in a stationary state as second coordinates, and a tracking processing unit that tracks the human body on the basis of the first coordinates and the second coordinates.

With this configuration, the first coordinates are detected when the human body as a detection target is moving and the second coordinates are detected when the human body is stationary. Thus, continuous tracking of the human body can be achieved.

A human body tracking method according to an aspect of the present disclosure includes a transmitting/receiving step of receiving a reflected wave of a transmitted radio wave, and a processing step of estimating a location of a human body on the basis of an IF signal output in the transmitting/receiving step. The processing step includes a first step of detecting coordinates of a moving object as first coordinates, a second step of detecting coordinates of at least the human body in a stationary state as second coordinates, and a third step of tracking the human body on the basis of the first coordinates and the second coordinates.

With this configuration, the first coordinates are detected when the human body as a detection target is moving and the second coordinates are detected when the human body is stationary. Thus, continuous tracking of the human body can be achieved.

### Advantageous Effects

According to the present disclosure, a human body tracking device and a human body tracking method capable of improving the accuracy in tracking of a human body can be implemented.

### BRIEF DESCRIPTION OF DRAWINGS

FIG. 1 is a block diagram illustrating a schematic configuration of a human body tracking device according to an embodiment.

FIG. 2 is a conceptual diagram illustrating locations of objects.

FIG. 3 is a diagram illustrating an example of a specific procedure of a location estimation method using an FMCW radar.

FIG. 4 is a conceptual diagram illustrating the positional relationship between the FMCW radar and a detection target.

FIG. 5 is a flowchart illustrating an example of a human body tracking process in a human body tracking device according to an embodiment.

FIG. 6 is a block diagram illustrating a schematic configuration of a first detection unit.

FIG. 7 is a sub-flowchart illustrating an example of a first coordinates detection process.

FIG. 8 is a block diagram illustrating a schematic configuration of a second detection unit.

FIG. 9 is a sub-flowchart illustrating an example of a second coordinates detection process.

FIG. 10 is a conceptual diagram for explaining an example of a power distribution calculation procedure.

FIG. 11 is a conceptual diagram illustrating two-dimensional data including amplitude variance for each distance and each angle.

FIG. 12 is a conceptual diagram illustrating two-dimensional data including degrees of correlation between an amplitude component and a phase component for each distance and each angle.

FIG. 13 is a conceptual diagram illustrating two-dimensional data including results of element-wise product operations for each distance and each angle.

FIG. 14A is a conceptual diagram illustrating locations of objects obtained by power distribution.

FIG. 14B is a conceptual diagram illustrating the location of an object obtained by a result of an element-wise product operation.

FIG. 15A is a conceptual diagram illustrating changes with time of first coordinates.

FIG. 15B is a conceptual diagram illustrating changes with time of the first coordinates.

FIG. 16A is a conceptual diagram illustrating changes with time of second coordinates.

FIG. 16B is a conceptual diagram illustrating changes with time of the second coordinates.

FIG. 17A is a conceptual diagram illustrating changes with time of coordinates indicating an estimated location of a human body.

FIG. 17B is a conceptual diagram illustrating changes with time of coordinates indicating an estimated location of the human body.

### DESCRIPTION OF EMBODIMENTS

Hereinafter, a human body tracking device and a human body tracking method according to an embodiment will be described in detail with reference to drawings. It should be noted that the present disclosure is not limited to an embodiment described below.

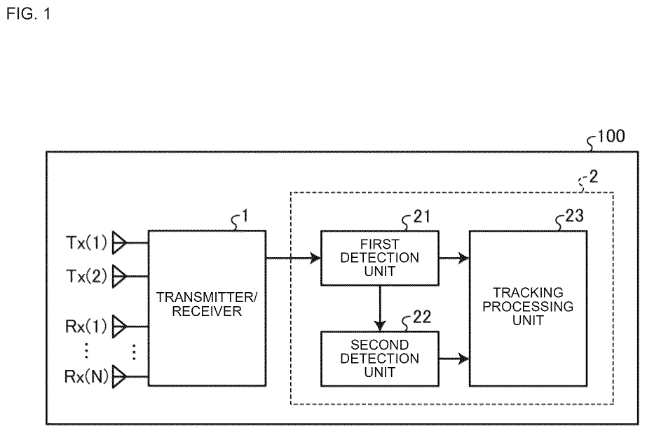

FIG. 1 is a block diagram illustrating a schematic configuration of a human body tracking device according to an embodiment. In the present disclosure, a human body tracking device 100 is a RAdio Detection And Ranging (RADAR) that transmits/receives radio waves and tracks a human body. Furthermore, in this embodiment, the human body tracking device 100 is explained as a radar of Frequency Modulated Continuous Wave (FMCW) type. Since FMCW radars are well known, detailed explanation may be omitted.

The human body tracking device 100 according to this embodiment includes a transmitter/receiver 1 and a processor 2. The transmitter/receiver 1 includes transmission antennas Tx(m) (m represents a natural number from 1 to the number M of transmission antennas) and reception antennas Rx(n) (n represents a number from 1 to the number N of reception antennas). In FIG. 1, an example in which two transmission antennas Tx(1) and Tx(2) are provided is illustrated. Furthermore, in FIG. 1, an example in which N reception antennas Rx(1), . . . , and Rx(N) are provided is illustrated. The present disclosure is not intended to be limited by the number of transmission antennas and the number of reception antennas.

The transmitter/receiver 1 transmits radio waves in, for example, millimeter-wave bands or microwave bands and receives reflected waves Rx of the corresponding radio waves. The processor 2 estimates the location of a human body on the basis of an IF signal output from the transmitter/receiver 1.

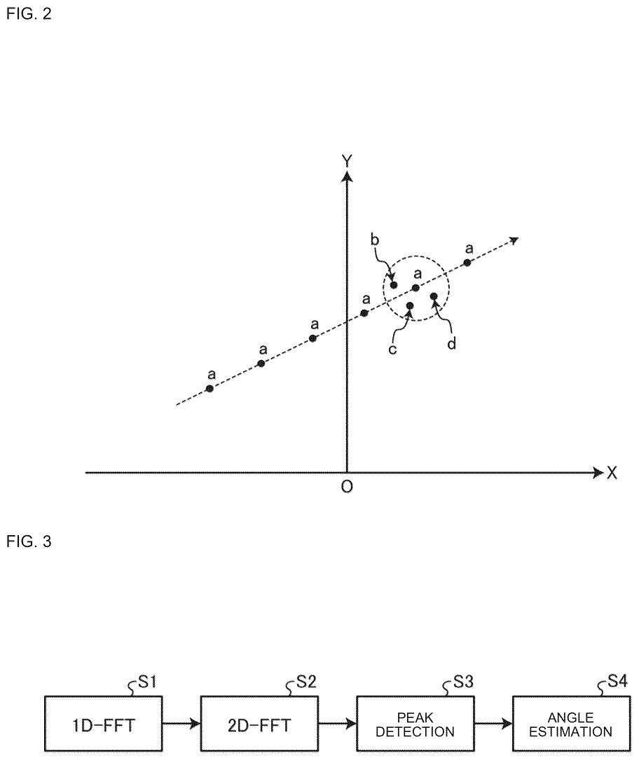

FIG. 2 is a conceptual diagram illustrating locations of objects. In FIG. 2, the origin O on the XY-plane is defined as the location of the human body tracking device 100. An X-direction represents a direction in which the reception antennas Rx(1), . . . , and Rx(N) are arranged, and a Y-direction represents a direction that is orthogonal to the direction in which the reception antennas Rx(1), . . . , and Rx(N) are arranged. Coordinates a illustrated in FIG. 2 indicate the location of a human body, and coordinates b, c, and d indicate the locations of stationary objects. The human body indicated by the coordinates a is moving towards a direction indicated by a broken line arrow.

FIG. 3 is a diagram illustrating an example of a specific procedure of a location estimation method using the FMCW radar. In typical FMCW radars, a distance Fast Fourier Transform (FFT) process (hereinafter, also referred to as a “1D-FFT process”) is performed for an IF signal output from a transmitter/receiver so that distance information is acquired (step S1). After that, a speed FFT process (hereinafter, also referred to as a “2D-FFT process”) is performed for a complex signal obtained by the 1D-FFT process (step S2).

Then, a peak detection process is performed for a result of the 2D-FFT process so that speed information is acquired (step S3). After that, an angle estimation process is performed on the basis of the distance information and the speed information so that angle information of an object is acquired (step S4).

In the typical location estimation method using an FMCW radar described above, detection accuracy deteriorates when a plurality of objects are close to each other. Specifically, as illustrated in FIG. 2, in the case where the human body indicated by the coordinates a is moving closer to the stationary objects indicated by the coordinates b, c, and d, the stationary objects may be falsely determined to be the human body. If a stationary object is falsely determined to be the human body, the coordinates of the stationary object may be determined to be the location of the person whose coordinates should be tracked.

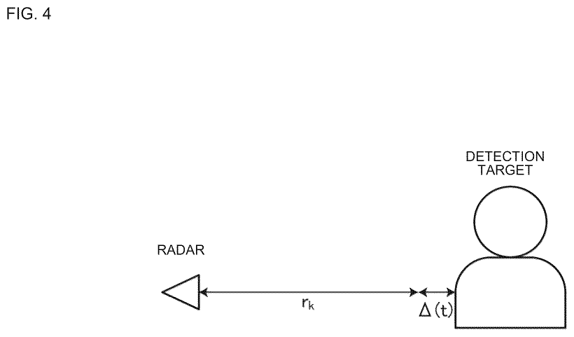

FIG. 4 is a conceptual diagram illustrating the positional relationship between the FMCW radar and a detection target. In FIG. 4, rₖ represents a distance in the k-th (k represents an integer from 0 to N−1, N represents the number of samples (the number of reception antennas)) frequency bin, and Δ(t) represents a minute variation component for the distance rₖ at time t.

An IF signal x(t,n) obtained by the transmitter/receiver of the radar is expressed by Equation (1) mentioned below. In Equation (1) mentioned below, M(t,r) represents an amplitude component at time t and distance r, and distance r indicates the distance between a radar 11 and a biological information acquisition target, and P(t,r) represents a phase component at time t and distance r.

$$\begin{matrix}[ Math . 1 ] & \\ x ( t , n ) = {\sum }_{r} M ( t , r ) \cdot cos 2 π {f}_{r} n + P ( t , r ) & ( 1 )\end{matrix}$$

The amplitude component M(t,r) and the phase component P(t,r) are extracted by performing Discrete Fourier Transform (DFT) on Equation (1) mentioned above. The amplitude component M(t,r) is expressed by Equation (2) mentioned below. The phase component P(t,r) is expressed by Equation (3) mentioned below. M₀ in Equation (2) mentioned below represents the amplitude of a transmission signal.

$$\begin{matrix}[ Math . 2 ] & \\ M ( t , r ) = \frac{{M}_{0}}{4 {π ( {r}_{k} + Δ ( t ) )}^{2}} & ( 2 )\end{matrix}$$

$$\begin{matrix}[ Math . 3 ] & \\ P ( t , r ) = \frac{4 π {f}_{c}}{c} ( {r}_{k} - Δ ( t ) ) & ( 3 )\end{matrix}$$

By performing Taylor expansion of the amplitude component M(t,r) expressed by Equation (2) mentioned above under the condition Δ(t)<<rₖ, Equation (4) mentioned below is obtained.

$$\begin{matrix}[ Math . 4 ] & \\ M ( t , r ) = \frac{{M}_{0}}{4 π {r}_{k}^{2}} ( 1 - 2 \cdot \frac{Δ ( t )}{{r}_{k}} ) & ( 4 )\end{matrix}$$

As expressed by Equation (3) and Equation (4) mentioned above, each of the amplitude component M(t,r) and the phase component P(t,r) of the IF signal x(t,n) expressed by Equation (1) mentioned above is proportional to the minute variation component Δ(t). In the case where the detection target illustrated in FIG. 4 is a human body, the minute variation component Δ(t) contains a body surface displacement based on a vital sign such as the heart rate of the human body. In other words, both the amplitude component M(t,r) and the phase component P(t,r) of the IF signal x(t,n) expressed by Equation (1) mentioned above change depending on a body surface displacement of the human body.

In the case where the human body is stationary, a body surface displacement of the human body is dominant in the minute variation component Δ(t). Thus, in the case where a detection target is a stationary human body, the degree of correlation between the amplitude component M(t,r) and the phase component P(t,r) is high.

In contrast, in the case where the detection target is a stationary object other than human bodies, the degree of correlation between the amplitude component M(t,r) and the phase component P(t,r) is low. Furthermore, even in the case where the detection target is a human body, when the human body is moving, the proportion of a body surface displacement of the human body in the minute variation component Δ(t) is relatively low compared to the case where the human body is stationary. By using the characteristics, a difference between at least a stationary human body and other cases (a stationary object other than the human body and the human body in a moving state) can be identified. In particular, by leveraging the specific physical characteristics of vital signs (e.g., respiration and heartbeat) inherent to a living body. By calculating the correlation between these specific amplitude and phase variances, the processor 2 functions as a specific biological signal filter. This allows the system to actively suppress high-power reflections from static non-living objects while amplifying the signal of the stationary human without requiring reference background data.

A detailed configuration and an operation of the processor 2 of the human body tracking device 100 according to an embodiment will be described below. An example of a configuration and an operation under the assumption of two-dimensional coordinates will be illustrated.

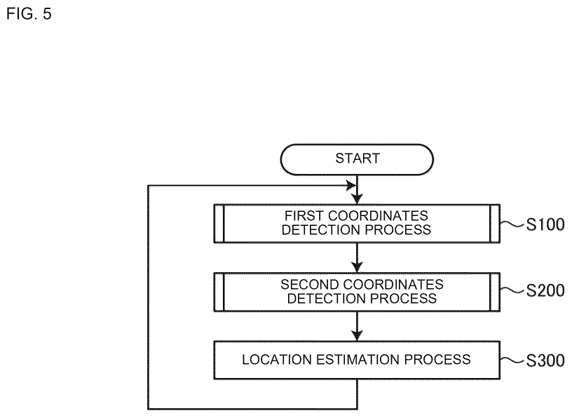

Referring back to FIG. 1, the processor 2 includes a first detection unit 21, a second detection unit 22, and a tracking processing unit 23. FIG. 5 is a flowchart illustrating an example of a human body tracking process in a human body tracking device according to an embodiment. As used herein, “unit” refers to circuitry that may be configured via the execution of computer readable instructions, and the circuitry may include one or more local processors (e.g., CPU's), and/or one or more remote processors, such as a cloud computing resource, or any combination thereof. The tracking processing unit 23 may also include a non-transitory computer-readable medium storing the program that, when executed by a processor, causes the processor to perform to the process illustrated in FIG. 5.

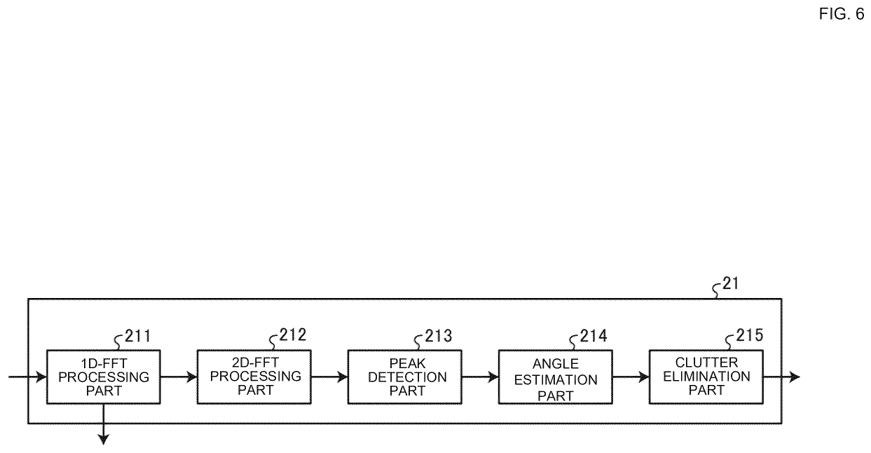

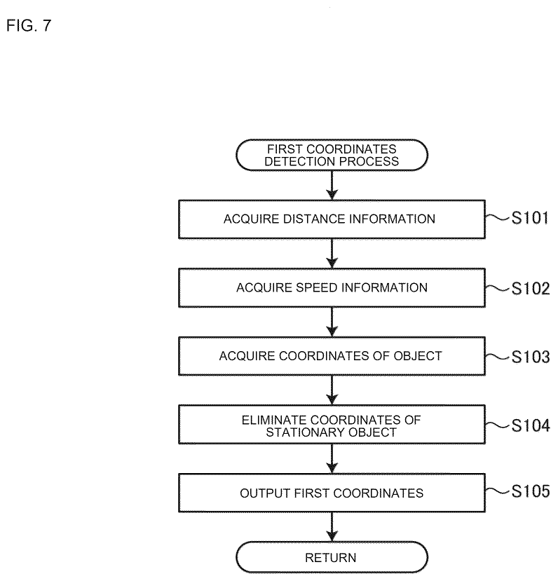

First, a configuration and an operation of the first detection unit 21 will be described. The first detection unit 21 detects coordinates of a moving object as first coordinates (step S100). FIG. 6 is a block diagram illustrating a schematic configuration of the first detection unit. The first detection unit 21 includes a 1D-FFT processing part 211, a 2D-FFT processing part 212, a peak detection part 213, an angle estimation part 214, and a clutter elimination part 215. FIG. 7 is a sub-flowchart illustrating an example of a first coordinates detection process. In the present disclosure, reflection from a stationary object is also referred to as “clutter.”

The 1D-FFT processing part 211 performs a 1D-FFT process on an IF signal output from the transmitter/receiver 1 so that distance information is acquired (step S101).

The 2D-FFT processing part 212 performs a 2D-FFT process on a complex signal obtained by the 1D-FFT process.

The peak detection part 213 performs a peak detection process on a result of the 2D-FFT process so that speed information is acquired (step S102).

The angle estimation part 214 performs an angle estimation process on the basis of the distance information and the speed information so that angle information of an object is acquired and the coordinates of the object are acquired (step S103).

The clutter elimination part 215 determines, based on the speed information acquired by the peak detection part 213, whether the coordinates of the object acquired by the angle estimation part 214 are coordinates of a stationary object or coordinates of a moving object. Then, the clutter elimination part 215 eliminates coordinates of a stationary object from among coordinates of objects acquired by the angle estimation part 214 (step S104), outputs coordinates of a moving object as the first coordinates (step S105), and returns to the human body tracking process illustrated in FIG. 5. In other words, the clutter elimination part 215 serves to reduce false positives in the first detection path. By filtering out coordinates with zero velocity (or velocity below a noise threshold), the processor 2 ensures that the first detection unit 21 is dedicated exclusively to motion tracking. This creates a clear functional separation between the “moving object” processing path and the “stationary vital sign” processing path, preventing the processing of static clutter in the motion tracking.

Next, a configuration and an operation of the second detection unit 22 will be described. In the present disclosure, the second detection unit 22 detects coordinates of a stationary human body as second coordinates (step S200).

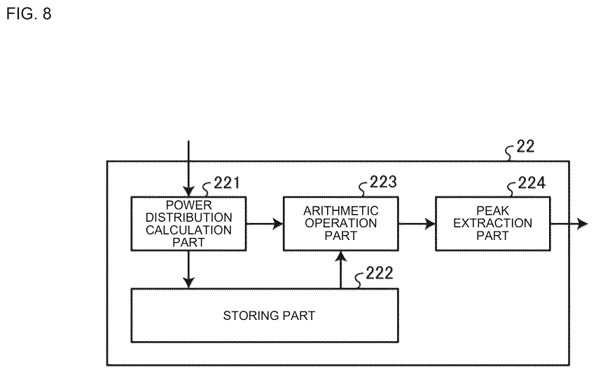

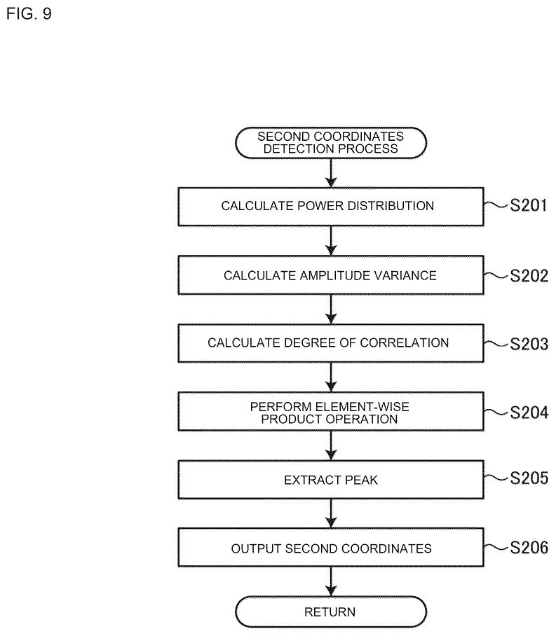

FIG. 8 is a block diagram illustrating a schematic configuration of the second detection unit. The second detection unit 22 includes a power distribution calculation part 221, a storing part 222, an arithmetic operation part 223, and a peak extraction part 224. FIG. 9 is a sub-flowchart illustrating an example of a second coordinates detection process.

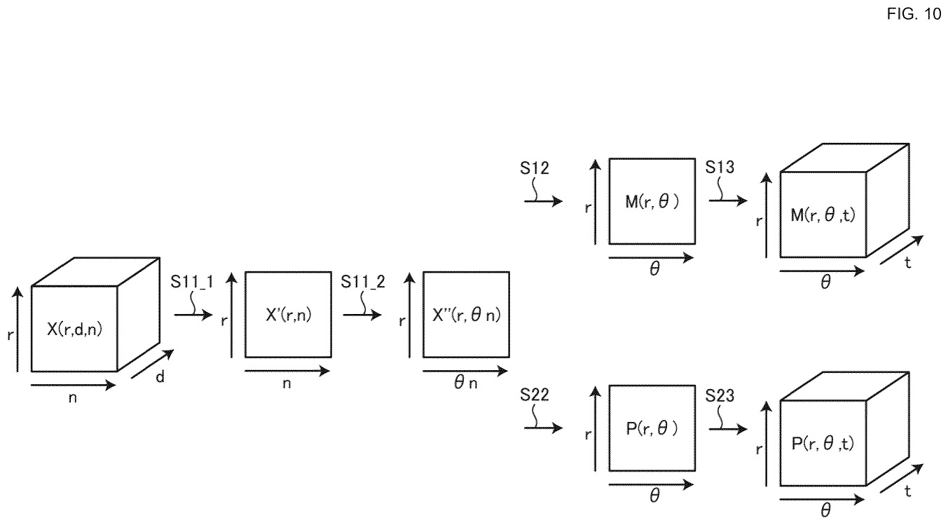

The power distribution calculation part 221 calculates power distribution on the basis of a signal X(r,d,n) obtained by the 1D-FFT process (step S201). FIG. 10 is a conceptual diagram for explaining an example of a power distribution calculating procedure. The signal X(r,d,n) obtained by the 1D-FFT process is three-dimensional data including a distance direction (hereinafter, also referred to as an “r-direction”), a speed direction (hereinafter, also referred to as a “d-direction”), and a direction in which reception antennas are arranged (hereinafter, also referred to as an “n-direction”).

The power distribution calculation part 221 performs an averaging process in the d-direction on the signal X(r,d,n), which is obtained by the 1D-FFT process, so that two-dimensional data X′(r,n) is generated (step S11_1).

Then, the power distribution calculation part 221 performs an angle estimation process based on the two-dimensional data X′(r,n) so that two-dimensional data X″(r,θn) corresponding to the power distribution is acquired (step S11_2). On represents an arbitrary angle. Thus, amplitude and phase components for each distance and each angle can be obtained. The angle estimation process performed by the power distribution calculation part 221 may be performed in a method similar to that for the angle estimation process performed by the angle estimation part 214 of the first detection unit 21 or may be performed in a method different from that for the angle estimation process performed by the angle estimation part 214 of the first detection unit 21. The present disclosure is not intended to be limited by a specific method for the angle estimation process performed by the power distribution calculation part 221.

Then, the power distribution calculation part 221 generates two-dimensional data M(r,θ) including amplitude components for each distance and each angle (step S12) and stores the generated data into the storing part 222 (step S13). Thus, three-dimensional data M(r,θ,t) including amplitude components for each distance and each angle in a time direction (hereinafter, also referred to as a “t-direction”) is generated. Furthermore, the power distribution calculation part 221 generates two-dimensional data P(r,θ) including phase components for each distance and each angle (step S22) and stores the generated data into the storing part 222 (step S23). Thus, three-dimensional data P(r,θ,t) including phase components for each distance and each angle in the t-direction is generated.

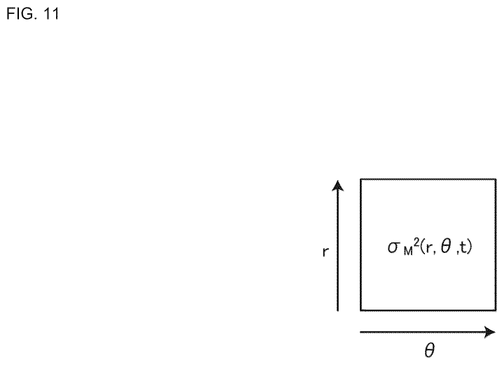

The arithmetic operation part 223 calculates amplitude variance during a period T (for example, 1 sec) based on the current time and generates two-dimensional data σ_{M}²(r,θ,t) including amplitude variance for each distance and each angle (step S202). The amplitude variance is expressed by Equation (5) mentioned below. FIG. 11 is a conceptual diagram illustrating two-dimensional data including amplitude variance for each distance and each angle.

$$\begin{matrix}[ Math . 5 ] & \\ {σ}_{M}^{2} ( r , θ , t ) = \frac{1}{T} {\int }_{0}^{T} {\{ M ( r , θ , t ) - \frac{1}{T} {\int }_{0}^{T} M ( r , θ , t ) dt \}}^{2} dt & ( 5 )\end{matrix}$$

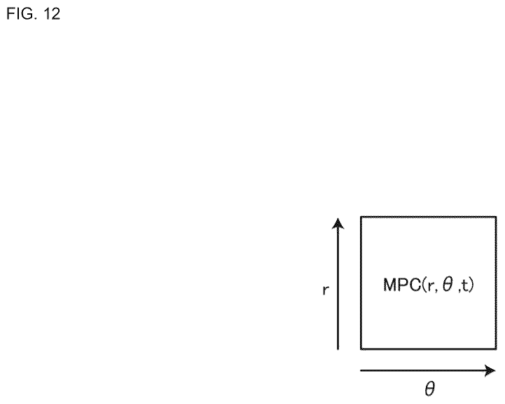

Furthermore, the arithmetic operation part 223 calculates degrees of correlation between amplitude components and phase components during the period T (for example, 1 second) based on the current time and generates two-dimensional data MPC(r,θ,t) including the degrees of correlation between the amplitude components and the phase components for each distance and each angle (step S203). The degrees of correlation are expressed by Equation (6) mentioned below. FIG. 12 is a conceptual diagram illustrating two-dimensional data including the degrees of correlation between the amplitude components and the phase components for each distance and each angle.

$$\begin{matrix}[ Math . 6 ] & \\ MPC ( r , θ , t ) = \vert \frac{1}{T} {\int }_{0}^{T} [ \{ M ( r , θ , t ) - \frac{1}{T} {\int }_{0}^{T} M ( r , θ , t ) dt \} \times \{ P ( r , θ , t ) - \frac{1}{T} {\int }_{0}^{T} P ( r , θ , t ) dt \} ] dt \vert & ( 6 )\end{matrix}$$

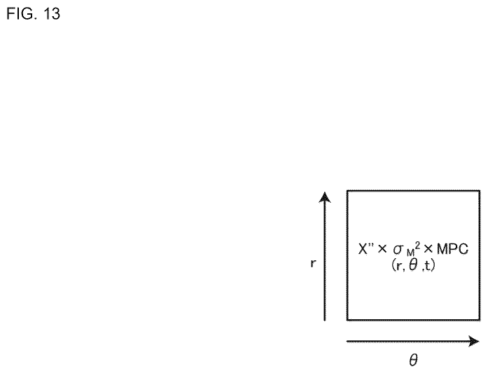

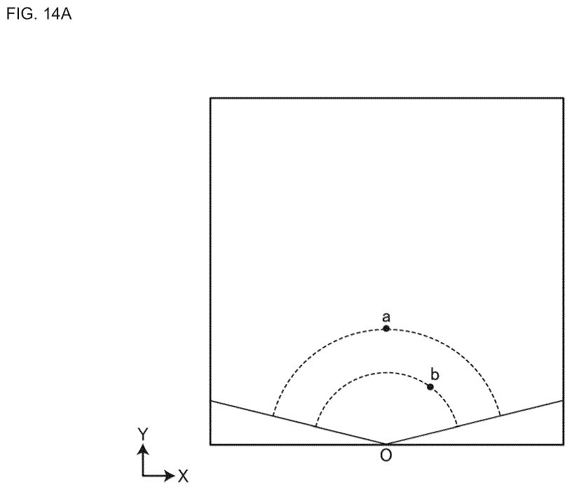

Then, the arithmetic operation part 223 performs an element-wise product operation process for the power distribution represented by power strength for each distance and each angle, the amplitude variance for each distance and each angle, and the degrees of correlation for each distance and each angle (step S204). By calculating the element-wise product of the power distribution (X″), the amplitude variance (σ_{M}²), and the correlation degree (MPC), the processor effectively acts as a biological filter. The power distribution identifies object presence; the amplitude variance identifies vibration; and the correlation degree confirms that the vibration is biological (coupled amplitude and phase modulation). FIG. 13 is a conceptual diagram illustrating two-dimensional data including results of element-wise product operations for each distance and each angle.

The peak extraction part 224 performs a well-known peak extraction process for the results of the element-wise product operations (X″×σ_{M}²×MPC(r,θ,t)) in the arithmetic operation part 223 (step S205). As the well-known peak extraction process, for example, a Cell Averaging Constant False Alarm Rate (CA-CFAR) process is illustrated. Detailed explanation for the CA-CFAR process is omitted here.

Specifically, the peak extraction part 224 performs the CA-CFAR process for the results of the element-wise product operations (X″×σ_{M}²×MPC(r,θ,t)) illustrated in FIG. 13. Then, the peak extraction part 224 acquires coordinates at which the element-wise product operation result is the local maximum value, outputs the coordinates as the second coordinates (step S206), and returns to the human body tracking process illustrated in FIG. 5. The method for acquiring the second coordinates is not limited to the method described above. For example, an aspect may be employed in which coordinates at which the element-wise product operation result is equal to or more than a predetermined threshold value are acquired as the second coordinates or an aspect may be employed in which coordinates at which the element-wise product operation result is the maximum value are acquired as the second coordinates.

The peak extraction process performed by the peak extraction part 224 is not limited to the CA-CFAR process. For example, an aspect may be employed in which coordinates at which the element-wise product operation result is equal to or more than a predetermined threshold value are output as the second coordinates. As the peak extraction process performed in the human body tracking process by the human body tracking device 100 according to the present disclosure, a well-known peak extraction process including the CA-CFAR process described above may be used.

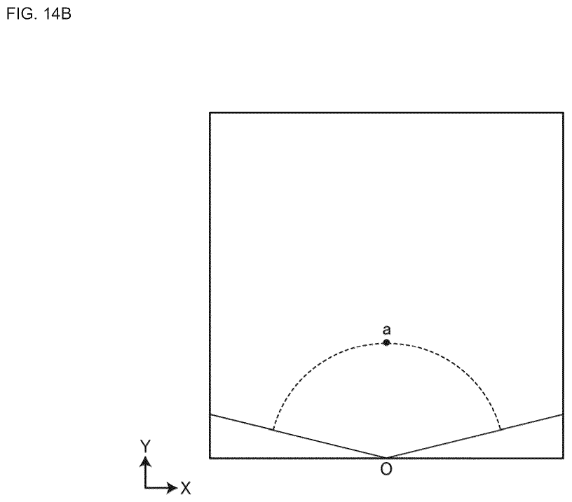

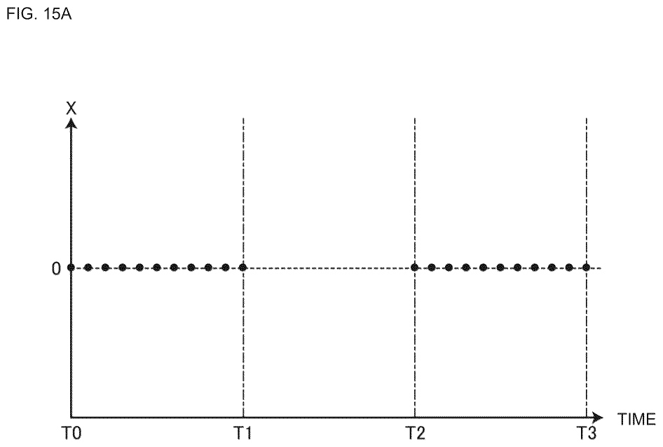

FIG. 14A is a conceptual diagram illustrating locations of objects obtained by power distribution. FIG. 14B is a conceptual diagram illustrating the location of an object obtained by a result of an element-wise product operation.

In FIGS. 14A and 14B, the origin O on the XY-plane is defined as the location of the human body tracking device 100, the X-direction represents the direction in which the reception antennas Rx(1), . . . , and Rx(N) are arranged, and the Y-direction represents the direction that is orthogonal to the direction in which the reception antennas Rx(1), . . . , and Rx(N) are arranged, as in FIG. 2. Coordinates a illustrated in FIGS. 14A and 14B indicate the location of a human body, and coordinates b indicate the location of a stationary object.

In the case of a human body, as described above, a body surface displacement based on a vital sign such as heart rate is contained in the minute variation component Δ(t) illustrated in FIG. 2. In particular, in the case where the human body is stationary, a body surface displacement of the human body is dominant in the minute variation component Δ(t). Thus, the degree of correlation between an amplitude component and a phase component is high. Consequently, the result of an element-wise product operation in the arithmetic operation part 223 is large. Accordingly, as illustrated in FIG. 14B, the coordinates a indicating the location of the human body are extracted as the second coordinates.

In the case of a stationary object, a body surface displacement based on a vital sign such as the heart rate of a human body is not contained in the minute variation component Δ(t) illustrated in FIG. 2. Thus, the degree of correlation between an amplitude component and a phase component is low, and the result of an element-wise product operation in the arithmetic operation part 223 is small. Accordingly, the stationary object indicated by the coordinates b in FIG. 14A is excluded in the peak extraction process by the peak extraction part 224, as illustrated in FIG. 14B.

In the case where the human body is moving, a body surface displacement based on a vital sign such as heart rate is relatively small with respect to a variation component. Therefore, the human body is not necessarily extracted as the second coordinates.

Referring back to FIG. 5, the first coordinates detected by the first detection unit 21 and the second coordinates detected by the second detection unit 22 are input to the tracking processing unit 23. The tracking processing unit 23 performs a well-known location estimation process for the first coordinates detected by the first detection unit 21 and the second coordinates detected by the second detection unit 22 (step S300). As the well-known location estimation process, for example, an extended Kalman filter is illustrated. Detailed explanation for the extended Kalman filter is omitted here.

Specifically, the tracking processing unit 23 applies the extended Kalman filter to the first coordinates detected by the first detection unit 21 and the second coordinates detected by the second detection unit 22 so that the location of the human body is estimated. In particular, the tracking processing unit 23 dynamically weighs the input from the first detection unit 21 (motion tracking) and the second detection unit 22 (variance-based tracking). This dual-path architecture optimizes the radar's tracking continuity. When the target slows down, the reliability (weight) of the first coordinates decreases as the Doppler shift approaches zero, while the detection of the second coordinates (vital sign based) increase. The tracking processing unit 23 correlates the disappearance of the first coordinates with the appearance of the second coordinates to determine that the moving object has transitioned to a stationary state, thereby preventing track fragmentation.

The location estimation process performed by the tracking processing unit 23 is not limited to the extended Kalman filter. For example, an aspect may be employed in which a particle filter is applied to the first coordinates detected by the first detection unit 21 and the second coordinates detected by the second detection unit 22 so that the location of the human body is estimated. As the location estimation process performed in the human body tracking process by the human body tracking device 100 according to the present disclosure, a well-known location estimation process including the extended Kalman filter or the particle filter described above may be used.

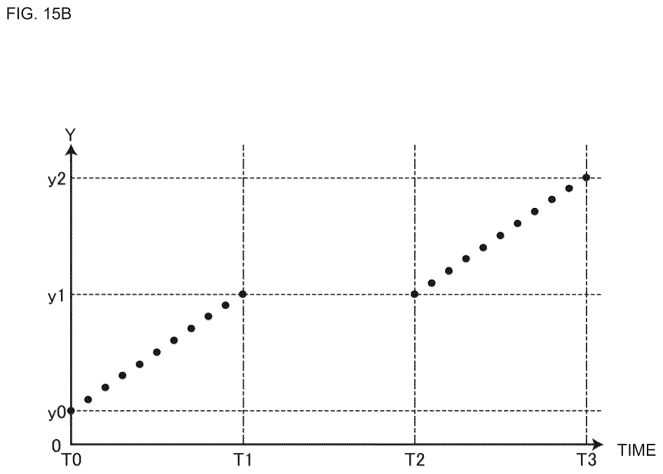

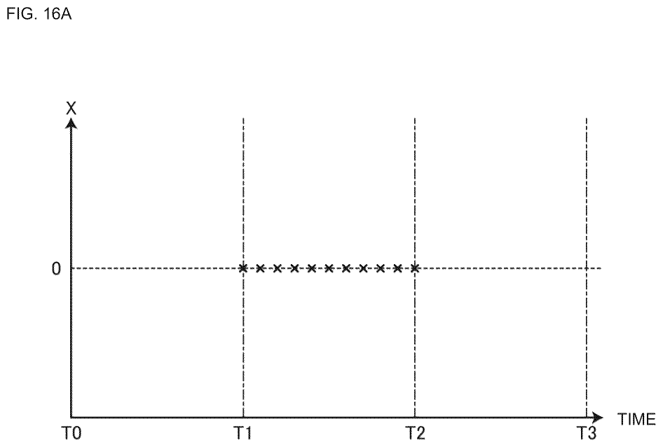

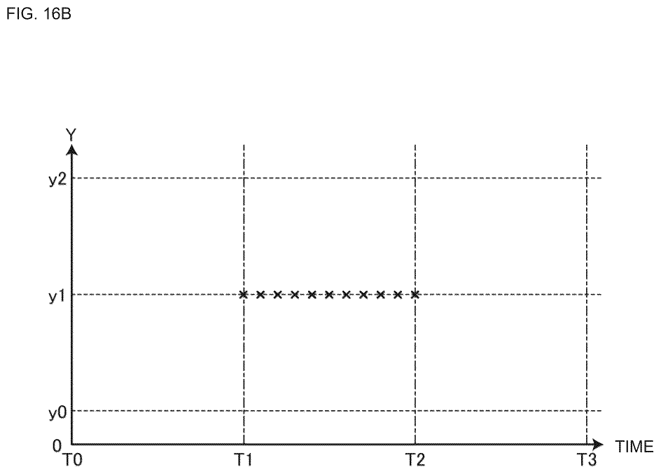

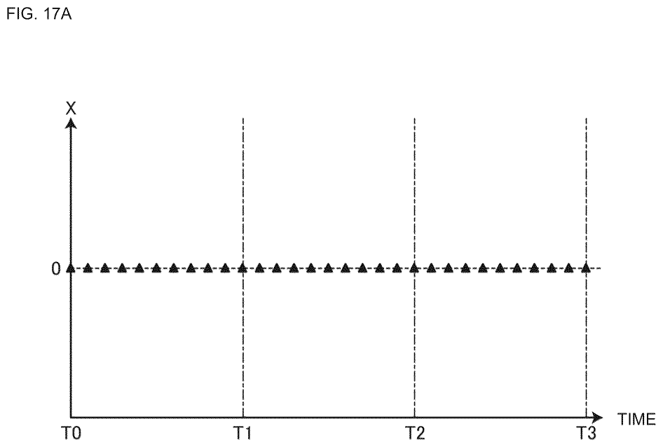

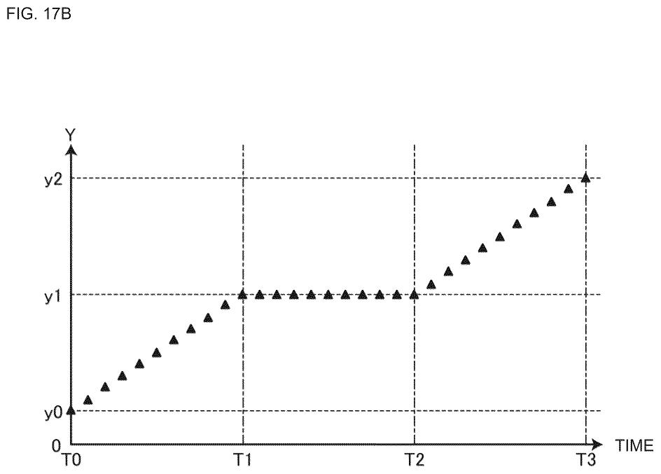

FIGS. 15A and 15B are conceptual diagrams illustrating changes with time of the first coordinates. FIGS. 16A and 16B are conceptual diagrams illustrating changes with time of the second coordinates. FIGS. 17A and 17B are conceptual diagrams illustrating changes with time of coordinates indicating an estimated location of the human body. An example in which the human body moves at a uniform speed from coordinates (0,y0) at time T0 to coordinates (0,y1) at time T1, stays stationary at the coordinates (0,y1) from the time T1 to time T2, and then moves at a uniform speed from the coordinates (0,y1) at the time T2 to coordinates (0,y2) at time T3 is illustrated.

As illustrated in FIGS. 15A and 15B, during the period from the time T0 to the time T1 and the period from the time T2 to the time T3 in which the human body is moving, coordinates of the moving object detected by the first detection unit 21 are input as the first coordinates to the tracking processing unit 23. Coordinates of stationary objects are eliminated by the clutter elimination part 215 of the first detection unit 21.

In contrast, as illustrated in FIGS. 16A and 16B, during the period from the time T1 to the time T2 in which the human body is stationary, the coordinates of the human body detected by the second detection unit 22 are input as the second coordinates to the tracking processing unit 23. Coordinates of objects (stationary objects other than the human body and the human body in a moving state) other than at least the stationary human body are excluded by the peak extraction process in the peak extraction part 224 of the second detection unit 22.

Then, by the location estimation process performed by the tracking processing unit 23, as illustrated in FIGS. 17A and 17B, during the entire period from the time T0 to the time T3 including the period from the time T0 to the time T1 in which the human body is moving, the period from the time T1 to the time T2 in which the human body is stationary, and the period from the time T2 to the time T3 in which the human body is moving, tracking of the human body can be achieved. Thus, with the configuration in which a vital sign is acquired based on a body surface displacement of the human body such as heart rate, continuous acquisition of vital signs can be achieved.

The embodiments described above are intended to facilitate understanding of the present disclosure and are not to be interpreted as limiting the present invention. The present disclosure can be modified or improved without departing from the gist of the disclosure, and the present disclosure encompasses equivalents thereof.

The present disclosure may include the following configurations as described above or instead of the above.
(1) A human body tracking device according to an aspect of the present disclosure comprising:
a transmitter/receiver that receives a reflected wave of a transmitted radio wave; and
a processor that estimates a location of a human body on the basis of an IF signal output from the transmitter/receiver, wherein the processor includes a first detection unit that detects coordinates of a moving object as first coordinates, a second detection unit that detects coordinates of at least the human body in a stationary state as second coordinates, and a tracking processing unit that tracks the human body on the basis of the first coordinates and the second coordinates.

With this configuration, the first coordinates are detected when the human body as a detection target is moving and the second coordinates are detected when the human body is stationary. Thus, continuous tracking of the human body can be achieved.
(2) The human body tracking device according to (1) mentioned above, wherein the second detection unit extracts, as the second coordinates, coordinates determined based on an element-wise product of power distribution, amplitude variance, and the degree of correlation between an amplitude and a phase.

With this configuration, the coordinates of the human body with a high degree of correlation between the amplitude and the phase are extracted as the second coordinates. Thus, tracking of the human body in a stationary state can be prevented from being lost.
(3) The human body tracking device according to (1) or (2) mentioned above, wherein the first detection unit extracts the first coordinates by eliminating coordinates of a stationary object from among coordinates of objects.

With this configuration, the coordinates of the moving object are detected as the first coordinates. Thus, tracking of the human body in a moving state can be achieved.
(4) The human body tracking device according to any one of (1) to (3) mentioned above, wherein the tracking processing unit estimates the location of the human body by using an extended Kalman filter or a particle filter.

Thus, accuracy in estimation of the location of the human body can be increased.
(5) A human body tracking method according to an aspect of the present disclosure comprising:
a transmitting/receiving step of receiving a reflected wave of a transmitted radio wave; and
a processing step of estimating a location of a human body on the basis of an IF signal output in the transmitting/receiving step, wherein the processing step includes a first step of detecting coordinates of a moving object as first coordinates, a second step of detecting coordinates of at least the human body in a stationary state as second coordinates, and a third step of tracking the human body on the basis of the first coordinates and the second coordinates.

With this configuration, the first coordinates are detected when the human body as a detection target is moving and the second coordinates are detected when the human body is stationary. Thus, continuous tracking of the human body can be achieved.
(6) The human body tracking method according to (5) mentioned above, wherein in the second step, coordinates determined based on an element-wise product of power distribution, amplitude variance, and the degree of correlation between an amplitude and a phase are extracted as the second coordinates.

With this configuration, the coordinates of the human body with a high degree of correlation between the amplitude and the phase are extracted as the second coordinates. Thus, tracking of the human body in a stationary state can be prevented from being lost.
(7) The human body tracking method according to (5) or (6) mentioned above, wherein in the first step, the first coordinates are extracted by eliminating coordinates of a stationary object from among coordinates of objects.

With this configuration, the coordinates of the moving object are detected as the first coordinates. Thus, tracking of the human body in a moving state can be achieved.
(8) The human body tracking method according to any one of (5) to (7) mentioned above, wherein in the third step, the location of the human body is estimated by using an extended Kalman filter or a particle filter.

Thus, accuracy in estimation of the location of the human body can be increased.

According to the present disclosure, a human body tracking device and a human body tracking method capable of improving the accuracy in tracking of a human body can be implemented.

### REFERENCE SIGNS LIST

1 transmitter/receiver
2 processor
21 first detection unit
22 second detection unit
23 tracking processing unit
100 human body tracking device
211 1D-FFT processing part
212 2D-FFT processing part
213 peak detection part
214 angle estimation part
215 clutter elimination part
221 power distribution calculation part
222 storing part
223 arithmetic operation part
224 peak extraction part

1. A human body tracking device comprising:
a transmitter/receiver configured to transmit a radio wave and receive a reflected wave of the transmitted radio wave; and
a processor configured to estimate a location of a human body based on an intermediate frequency (IF) signal output from the transmitter/receiver,
wherein the processor is configured to execute:
a first detection process that detects coordinates of a moving object as first coordinates based on a Doppler shift of the IF signal,
a second detection process that detects coordinates of at least the human body in a stationary state as second coordinates based on a body surface displacement of the human body derived from the IF signal, and
a tracking process that tracks the human body based on the first coordinates and the second coordinates.

2. The human body tracking device according to claim 1,
wherein the second detection process extracts, as the second coordinates, coordinates determined based on an element-wise product of power distribution, amplitude variance, and the degree of correlation between an amplitude and a phase.

3. The human body tracking device according to claim 2, wherein the element-wise product is calculated using three-dimensional data generated from the IF signal, the three-dimensional data including a distance dimension, an angle dimension, and a time dimension.

4. The human body tracking device according to claim 2, wherein the degree of correlation indicates a likelihood of the body surface displacement of the human body caused by a vital sign.

5. The human body tracking device according to claim 1,
wherein the first detection process
extracts the first coordinates by eliminating coordinates of a stationary object from among detected coordinates of objects.

6. The human body tracking device according to claim 1,
wherein the tracking process estimates the location of the human body by using an extended Kalman filter or a particle filter to the first coordinates and the second coordinates.

7. The human body tracking device according to claim 1, wherein the tracking process further includes fusing the first coordinates and the second coordinates to maintain continuous tracking of the human body as the human body transitions between a moving state and the stationary state.

8. A human body tracking method comprising:
a transmitting a radio wave and receiving a reflected wave of the transmitted radio wave; and
estimating a location of a human body on the basis of an intermediate frequency (IF) signal output,
wherein estimating includes
detecting coordinates of a moving object as first coordinates based on a Doppler shift of the IF signal,
detecting coordinates of at least the human body in a stationary state as second coordinates based on a body surface displacement of the human body derived from the IF signal; and
tracking the human body on the basis of the first coordinates and the second coordinates.

9. The human body tracking method according to claim 8,
detecting the second coordinates includes determining, based on an element-wise product of power distribution, amplitude variance, and the degree of correlation between an amplitude and a phase are extracted as the second coordinates.

10. The human body tracking method according to claim 8, further comprising generating two-dimensional data including the amplitude variance for each distance and each angle.

11. The human body tracking method according to claim 8, wherein detecting the second coordinates includes calculating a variance of an amplitude of the IF signal over a predetermined period and a correlation between the amplitude and a phase of the IF signal.

12. The human body tracking method according to claim 8,
wherein in detecting the first coordinates includes eliminating coordinates of a stationary object from among coordinates of objects.

13. The human body tracking method according to claim 8,
tracking the location of the human body includes using an extended Kalman filter or a particle filter to the first coordinates and the second coordinates.

14. The human body tracking method according to claim 8, wherein tracking the human body further includes fusing the first coordinates and the second coordinates to maintain continuous tracking of the human body as the human body transitions between a moving state and the stationary state.

15. A non-transitory computer-readable medium storing a program that, when executed by a processor, causes the processor to perform operations comprising:
acquiring an intermediate frequency (IF) signal derived from a reflected wave of a radio wave;
detecting coordinates of a moving object as first coordinates based on the IF signal;
detecting coordinates of a human body in a stationary state as second coordinates based on the IF signal; and
tracking the human body based on the first coordinates and the second coordinates.

16. The non-transitory computer-readable medium according to claim 15, wherein detecting the second coordinates includes calculating an element-wise product of a power distribution of the IF signal, an amplitude variance of the IF signal, and a correlation between an amplitude and a phase of the IF signal.

17. The non-transitory computer-readable medium according to claim 15, wherein the operations further includes:
extracting a peak value from the calculated element-wise product; and
determining the second coordinates based on the extracted peak value.

18. The non-transitory computer-readable medium according to claim 15, wherein detecting the first coordinates includes removing data corresponding to objects identified as stationary clutter.

19. The non-transitory computer-readable medium according to claim 15, wherein the tracking includes fusing the first coordinates and the second coordinates to maintain continuous tracking of the human body as it transitions between a moving state and a stationary state.

20. The non-transitory computer-readable medium according to claim 15, wherein detecting the second coordinates is performed only when the first coordinates indicate a velocity below a predetermined threshold.
